(19) United States
(12) Patent Application Publication
(10) Pub. No.: US 2014/0356296 A1
(43) Pub. Date: Dec. 4, 2014

(54) Use Of Isosorbide Derivatives For Producing Cosmetic Preparations

(71) Applicants: Claudia Stoer, Dusseldorf (DE); Markus Dierker, Dusseldorf (DE); Claus Nieendick, Krefeld (DE); Werner Seipel, Hilden (DE); Ansgar Behler, Bottrop (DE); Daniela Prinz, Dormagen (DE); Catherine Breffa, Mannheim (DE); Markus Weissenegger, Dusseldorf (DE); Hans-Christian Raths, Monheim (DE)

(72) Inventors: Claudia Stoer, Dusseldorf (DE); Markus Dierker, Dusseldorf (DE); Claus Nieendick, Krefeld (DE); Werner Seipel, Hilden (DE); Ansgar Behler, Bottrop (DE); Daniela Prinz, Dormagen (DE); Catherine Breffa, Mannheim (DE); Markus Weissenegger, Dusseldorf (DE); Hans-Christian Raths, Monheim (DE)

(73) Assignee: BASF SE, Ludwigshafen (DE)

(21) Appl. No.: 14/345,736

(22) PCT Filed: Sep. 7, 2012
(86) PCT No.: PCT/EP2012/067481

§ 371 Date: Jun. 20, 2014

(30) Foreign Application Priority Data
Sep. 19, 2011 (EP) 11181837.3

(51) Int. Cl. A61K 8/49; A61Q 19/00; A61Q 5/00; C07D 493/04

(52) U.S. Cl. CPC A61K 8/4973; C07D 493/04; A61Q 19/00; A61Q 5/00; A61K 2800/48; A61K 2800/10; A61K 2800/26
USPC 424/47; 514/470; 549/464

(57) ABSTRACT

Described are isosorbide derivatives according to general formula

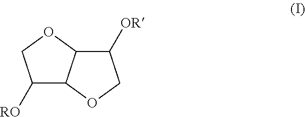

wherein R and R′, independently of one another, are: (i) hydrogen atom, or (ii) radical COR″, wherein R″ is linear or branched, saturated or unsaturated alkyl radical having 5 to 23 carbon atoms, or (iii) linear or branched, saturated or unsaturated alkyl radical having 6 to 22 carbon atoms, or (iv) radical CH₂—CHOH—R′″, wherein R′″ is linear or branched alkyl radical having 6 to 22 carbon atoms, or (v) radical (CH₂—CH₂O)ₙ—H and/or (CH₂—CH(CH₃)—O)ₘ—H, wherein n and m, independently of one another, are an integer or fraction from 1 to 10, or (vi) for radical SO₃OX, wherein X represents a sodium or ammonium ion, with the proviso that at most one of the radicals R and R′ is a hydrogen atom. The isosorbide derivatives are useful as components of cosmetic compositions.

### CROSS-REFERENCE TO RELATED APPLICATIONS

This application is the National Stage entry of PCT/EP2012/067481, filed on Sep. 7, 2012, which claims priority to European Application Number 11181837.3, filed on Sep. 19, 2011, which are incorporated herein by reference in their entireties.

### TECHNICAL FIELD

The present invention relates to the use of certain derivatives of isosorbide for producing cosmetic compositions and to cosmetic compositions which comprise these derivatives.

### BACKGROUND

In the field of cosmetic preparations for skin and haircare, a large number of requirements are imposed by the consumer: apart from the cleaning and care effects, which determine the intended use, value is placed on such differing parameters as best possible dermatological compatibility, good refatting properties, elegant appearance, optimal sensory impression and storage stability.

Preparations which are used for the cleaning and care of human skin and hair generally comprise, alongside a series of surface-active substances, especially oil bodies and water. The oil bodies/emollients used are, for example, hydrocarbons, ester oils and vegetable and animal oils/fats/waxes. In order to meet the high requirements of the market with regard to sensory properties and optimal dermatological compatibility, new oil bodies and emulsifier mixtures are being continually developed and tested. For the production of cosmetic preparations, a large number of natural and synthetic oils, for example almond oil or avocado oil, ester oils, ethers, alkyl carbonates, hydrocarbons, and also silicone oils are used. It is an essential task of the oil components, as well as the care effect, which is directly connected to skin greasing, to impart to the consumer a non-sticky, long-lasting feel of skin smoothness and suppleness which develops as quickly as possible.

As well as oil bodies, further constituents are also used in cosmetic compositions which, for example, influence the foaming behavior and/or the rheology, which serve as emulsifiers for the purpose of stably formulating aqueous and nonaqueous phases alongside one another, or which are able to impart further functionalities, e. g. a pearlescent effect.

There is therefore a constant need to provide new ingredients which are suitable for use in cosmetic compositions. It has now been found that derivatives of isosorbide can be used advantageously in cosmetic compositions.

### SUMMARY

Aspects of a first embodiment are directed to a method of producing a cosmetic composition, the method comprising mixing a cosmetic ingredient with an isosorbide derivative according to the general formula (I)

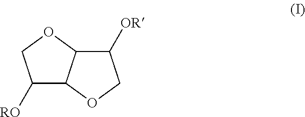

wherein R and R′, independently of one another, are: (i) a hydrogen atom, or (ii) a radical COR″, where R″ is a linear or branched, saturated or unsaturated alkyl radical having 5 to 23 carbon atoms, or (iii) a linear or branched, saturated or unsaturated alkyl radical having 6 to 22 carbon atoms, or (iv) a radical CH₂—CHOH—R″, where R′″ is a linear or branched alkyl radical having 6 to 22 carbon atoms, or (v) a radical (CH₂—CH₂O)ₙ—H and/or (CH₂—CH(CH₃)—O)ₘ—H and/or CH₂—CHOH—R′″, where n and m, independently of one another, can be an integer or fraction from 1 to 10, or (vi) for a radical SO₃X, where X represents a sodium or ammonium ion, with the proviso that at most one of the radicals R and R′ is a hydrogen atom.

In a second embodiment, the method the first embodiment is modified, wherein R═H and R′═COR″, wherein R″ is a linear or branched alkyl radical having 5 to 23 carbon atoms.

In a third embodiment, the method the first and second embodiments is modified, wherein R and R′, independently of one another, are radicals COR″, wherein R″ is a linear or branched, saturated or unsaturated alkyl radical having 5 to 23 carbon atoms.

In a fourth embodiment, the method of the first through third embodiments is modified, wherein R═H and R′ is a linear or branched alkyl radical having 6 to 22 carbon atoms.

In a fifth embodiment, the method of the first through fourth embodiments is modified, wherein R and R′, independently of one another, are a linear or branched alkyl radical having 6 to 22 carbon atoms.

In a sixth embodiment, the method of the first through fifth embodiment is modified, wherein R═H and R′ is a radical CH₂—CHOH—R″, wherein R′″ is a linear or branched alkyl radical having 6 to 22 carbon atoms.

In a seventh embodiment, the method of the first through sixth embodiments is modified, wherein R and R′, independently of one another, are a radical CH₂—CHOH—R′″, wherein R′″ is a linear or branched alkyl radical having 6 to 22 carbon atoms.

In an eighth embodiment, the method of the first through seventh embodiments is modified, wherein R═H and R′ is a radical (CH₂—CHO))ₙ— and/or (CH₂—CH(CH₃)—O)ₘ—, wherein n and m, independently of one another, are an integer or fraction from 1 to 10.

In a ninth embodiment, the method of the first through eighth embodiments is modified, wherein R and R′, independently of one another, are a radical (CH₂—CHO)ₙ—, where n is an integer or fraction from 1 to 10, or are a radical (CH₂—CH(CH₃)—O)ₘ—, where m is an integer or fraction from 1 to 10, or R and R′ contain both radicals (CH₂—CHO)ₙ and radicals (CH₂—CH(CH₃)—O)ₘ alongside one another.

In a tenth embodiment, the method of the first through ninth embodiments is modified, wherein R is a C6 to C22-alkyl radical and R′ is a radical SO₃X, wherein X represents a sodium or ammonium ion.

In an eleventh embodiment, the method of the first through tenth embodiments is modified, wherein at least two structurally different isosorbide derivatives of the general formula (I) are mixed alongside one another with the cosmetic ingredient.

In a twelfth embodiment, the method of the first through eleventh embodiments is modified, wherein either R or R′ is a hydrogen atom.

In a thirteenth embodiment, the method of the first through twelfth embodiments is modified, wherein R″ is a linear, saturated alkyl radical having 10 to 18.

In a fourteenth embodiment, the method of the third embodiment is modified, wherein R and R′, independently of one another, are linear, saturated alkyl radicals having 10 to 18 carbon atoms.

In a fifteenth embodiment, the method of the fourth embodiment is modified, wherein R′ is a linear or branched alkyl radical having 6 to 12 carbon atoms.

In a sixteenth embodiment, the method of the fifth embodiment is modified, wherein R and R′, independently of one another, are a linear or branched alkyl radical having 6 to 12 carbon atoms.

In a seventeenth embodiment, the method of the sixth embodiment is modified, wherein R′ is a radical CH₂—CHOH—R′″, wherein R′″ is a linear alkyl radical having 6 to 12.

In an eighteenth embodiment, the method of the seventh embodiment is modified, wherein R and R′, independently of one another, are a radical CH₂—CHOH—R″, wherein R′″ is a linear alkyl radical having 6 to 12.

In a nineteenth embodiment, the method of the eighth embodiment is modified, wherein R′ is a radical (CH₂—CHO)ₙ—, wherein n is an integer or fraction from 1 to 4.

In a twentieth embodiment, the method of the ninth embodiment is modified, wherein R and R′, independently of one another, are a radical (CH₂—CHO)ₙ—, wherein n is an integer or fraction from 1 to 4.

In a twenty-first embodiment, the method of the first through twentieth embodiments is modified, wherein the isosorbide derivative comprises from 0.5 to 30% by weight of the cosmetic composition.

In a twenty-second embodiment, the method of the first through twenty-first embodiments is modified, wherein the cosmetic composition is free from silicone oils.

In a twenty-third embodiment, the method of the first through twenty-second embodiments is modified, wherein the cosmetic composition is an aqueous cosmetic composition and wherein the cosmetic ingredient is selected from nonionic emulsifiers, hydrocarbons, astringents, dyes, fragrances, propellants, thickeners, and/or pearlizing agents.

In a twenty-fourth embodiment, the method of the first through twenty-third embodiments is modified, wherein the cosmetic composition is aqueous, and wherein the isosorbide derivative is effective to thicken the aqueous cosmetic composition.

In a twenty-fifth embodiment, the method of the first through twenty-third embodiments is modified, wherein the cosmetic composition is aqueous and wherein the isosorbide derivative is effective to emulsify the aqueous cosmetic composition.

In a twenty-sixth embodiment, the method of the first through twenty-third embodiments is modified, wherein the cosmetic composition is aqueous, and wherein the isosorbide derivative is effective to form an oil phase in the aqueous cosmetic composition.

In a twenty-seventh embodiment, the method of the first through twenty-third embodiments is modified, wherein the cosmetic composition is aqueous, and wherein the isosorbide derivative is effective to improve foam in the aqueous cosmetic composition.

Aspects of a twenty-eighth embodiment are directed to an isosorbide derivative according to the general formula (I)

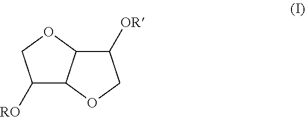

wherein R and R′, independently of one another, are a radical COR″, wherein R″ is a linear, saturated alkyl radical having 5 to 11 carbon atoms.

Aspects of a twenty-ninth embodiment are directed to a method of preparing a cosmetic composition, the method comprising mixing a cosmetic ingredient and an emollient comprising the isosorbide derivative of the twenty-eighth embodiment.

Aspects of a thirtieth embodiment are directed to anisosorbide derivative according to the general formula (I)

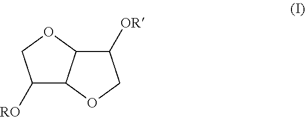

wherein one radical R or R′ is a hydrogen atom and the other radical R or R′ is a group COR″, wherein R″ is a linear, saturated alkyl radical having 15 to 19 carbon atoms.

Aspects of thirty-first embodiment are directed to a method of preparing pearlescent compositions, the method comprising mixing a cosmetic ingredient and the isosorbide derivative of the thirtieth embodiment.

Aspects of a thirty-second embodiment are directed to an isosorbide derivative according to the general formula (I)

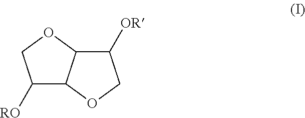

wherein one radical R or R′ is a hydrogen atom and the other radical R or R′ is a group COR″, wherein R″ is a linear, saturated or unsaturated alkyl radical having 9 to 15.

Aspects of a thirty-third embodiment are directed to a method of preparing aqueous cosmetic compositions, the method comprising mixing a cosmetic ingredient and a thickener comprising the isosorbide derivative of the thirty-second embodiment.

Aspects a thirty-fourth embodiment are directed to an isosorbide derivative according to the general formula (I)

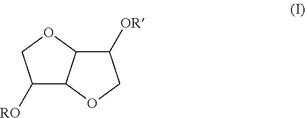

wherein R or R′ is a hydrogen atom and the other radical R or R′ is a group CH₂—CHOH—R″, wherein R″ is a linear alkyl radical having 12 to 18 carbon atoms.

Aspects of a thirty-fifth embodiment are directed to a method of preparing cosmetic compositions, the method comprising mixing a cosmetic ingredient and an emulsifier comprising the isosorbide derivative of the thirty-fourth embodiment.

Aspects of a thirty-sixth embodiment are directed to a method of boosting the foam of an aqueous composition, the method comprising mixing a foaming substance with a foam booster comprising an isosorbide derivative according to the general formula (I)

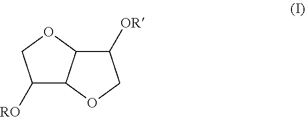

wherein R or R′ is a hydrogen atom and the other radical R or R′ is an alkyl group having 8 to 12 carbon atoms.

Aspects of a thirty-seventh embodiment are directed to a method of boosting the foam of an aqueous composition, the method comprising mixing a foaming substance with a foam booster comprising an isosorbide derivative according to the general formula (I)

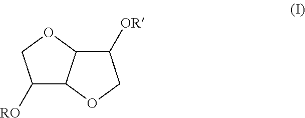

wherein R and R′ are a group ((CH₂—CH₂—O)ₙ—H and/or (CH₂—CH(CH₃)—O)ₘ—H, wherein n and m, independently of one another, are an integer or fraction from 1 to 10.

Aspects of a thirty-eighth embodiment are directed to a method of thickening an aqueous composition, the method comprising mixing an aqueous composition and a thickener comprising an isosorbide derivative according to the general formula (I)

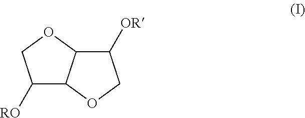

wherein one group R or R′ is an alkyl radical having 8 to 18 carbon atoms and the other group R or R′ is a radical SO₃X, with the proviso that R≠R′.

Aspects of a thirty-ninth embodiment are directed to a cosmetic composition comprising at least one water phase and one oil phase, and the isosorbide derivative of claim 28.

### BRIEF DESCRIPTION OF DRAWINGS

FIG. 1: A chart showing foam height for compositions prepared according to the Examples;

FIG. 2: A graph showing thickenability with sodium chloride of compositions prepared according to the Examples;

FIG. 3: Photographs showing foam quality;

FIG. 4: A chart showing Rota Foam Test results for compositions prepared according to the Examples; and

FIG. 5: A graph showing thickenability with sodium chloride for compositions prepared according to the Examples.

### DETAILED DESCRIPTION

Isosorbide (or 1,4′; 3,6-dianhydrosorbitol) is the anhydride of sorbitol. It can be obtained, for example, by heating sorbitol in the presence of concentrated sulfuric acid or hydrochloric acid. By means of methods known per se to the person skilled in the art, it is possible to obtain various derivatives of isosorbide, for example ethers, esters or salts.

Provided is the use of isosorbide derivatives according to the general formula (I)

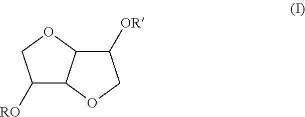

where R and R′, independently of one another, are:
   a hydrogen atom, or
   a radical COR″, where R″ is a linear or branched, saturated or unsaturated alkyl radical having 5 to 23 carbon atoms, or
   a linear or branched alkyl radical having 6 to 22 carbon atoms, or
   a radical CH₂—CHOH—R′″, where R′″ is a linear or branched, saturated or unsaturated alkyl radical having 6 to 22 carbon atoms, or
   a radical (CH₂—CH₂O)ₙ—H and/or (CH₂—CH(CH₃)—O)ₘ—H and/or CH₂—CHOH—R″, where n and m, independently of one another, can be an integer or fraction from 1 to 10, or
   for a radical SO₃X, where X represents a sodium or ammonium ion,
with the proviso that at most one of the radicals R and R′ is a hydrogen atom, for producing cosmetic compositions.

The general formula (I) also includes all stereoisomers of isosorbide, and any desired mixtures thereof. The non-derivatized isosorbides in which R and R′ are a hydrogen atom are explicitly excluded from protection. The general formula (I) otherwise includes all combinations of the radicals R and R′ among one another. In the formula (I), the group “(CH₂—CH(CH₃)—O)ₘ—H” always includes all conceivable positional isomers, individually or mixed, and also the group (CH(CH₃)—CH₂—O)ₘ—H.

Cosmetic compositions are to be understood here as meaning all compositions known to the person skilled in the art which are exclusively or primarily intended to be used externally on the human body or in its oral cavity for cleaning, care, protection, maintaining a good condition, perfuming, changing the appearance or for the purposes of influencing body odor.

The cosmetic compositions according to one or more embodiments can be in particular formulations for bodycare, e. g. a body milk, creams, lotions, sprayable emulsions, products for eliminating body odor etc. The hydrocarbons can also be used in surfactant-containing formulations such as e. g. foam and shower baths, hair shampoos and care rinses. Depending on the intended application, the cosmetic formulations comprise a series of further auxiliaries and additives, such as, for example, surfactants, further oil bodies, emulsifiers, pearlescent waxes, consistency regulators, thickeners, superfatting agents, stabilizers, polymers, fats, waxes, lecithins, phospholipids, biogenic active ingredients, UV light protection factors, antioxidants, deodorants, antiperspirants, antidandruff agents, film formers, swelling agents, insect repellents, self-tanning agents, tyrosine inhibitors (depigmentation agents), hydrotropes, solubilizers, preservatives, perfume oils, dyes etc., which are listed below by way of example.

Surfactants Surface-active substances which may be present are anionic, nonionic, cationic and/or amphoteric or zwitterionic surfactants. In one or more embodiments, in surfactant-containing cosmetic preparations, such as, for example, shower gels, foam baths, shampoos etc., at least one anionic surfactant is present. The fraction of surfactants here is usually about 1 to 30, specifically 5 to 25 and in particular 10 to 20% by weight.

Typical examples of anionic surfactants are soaps, alkylbenzenesulfonates, alkanesulfonates, olefinsulfonates, alkyl ether sulfonates, glycerol ether sulfonates, α-methyl ester sulfonates, sulfo fatty acids, alkyl sulfates, fatty alcohol ether sulfates, glycerol ether sulfates, fatty acid ether sulfates, hydroxy mixed ether sulfates, monoglyceride (ether) sulfates, fatty acid amide (ether) sulfates, mono- and dialkyl sulfosuccinates, mono- and dialkyl sulfosuccinamates, sulfotriglycerides, amide soaps, ether carboxylic acids and salts thereof, fatty acid isethionates, fatty acid sarcosinates, fatty acid taurides, N-acylamino acids, such as, for example, acyl lactylates, acyl tartrates, acyl glutamates and acyl aspartates, alkyl oligoglycoside sulfates, protein fatty acid condensates (in particular wheat-based vegetable products) and alkyl(ether) phosphates. If the anionic surfactants contain polyglycol ether chains, these can have a conventional homolog distribution, but specifically have a narrowed homolog distribution. Typical examples of nonionic surfactants are fatty alcohol polyglycol ethers, alkylphenol polyglycol ethers, fatty acid polyglycol esters, fatty acid amide polyglycol ethers, fatty amine polyglycol ethers, alkoxylated triglycerides, mixed ethers and mixed formals, optionally partially oxidized alk(en)yl oligoglycosides and glucuronic acid derivatives, fatty acid N-alkylglucamides, protein hydrolyzates (in particular wheat-based vegetable products), polyol fatty acid esters, sugar esters, sorbitan esters, polysorbates and amine oxides. If the nonionic surfactants contain polyglycol ether chains, these can have a conventional homolog distribution, but specifically have a narrowed homolog distribution. Typical examples of cationic surfactants are quaternary ammonium compounds, such as, for example, dimethyldistearylammonium chloride, and ester quats, in particular quaternized fatty acid trialkanolamine ester salts. Typical examples of amphoteric or zwitterionic surfactants are alkylbetaines, alkylamidobetaines, aminopropionates, aminoglycinates, imidazoliniumbetaines and sulfobetaines. Said surfactants are exclusively known compounds. As regards structure and preparation of these substances, reference may be made to relevant review works in this field. Typical examples of particularly suitable mild, i. e. particularly skin-compatible, surfacatants are fatty alcohol polyglycol ether sulfates, monoglyceride sulfates, mono- and/or dialkyl sulfosuccinates, fatty acid isethionates, fatty acid sarcosinates, fatty acid taurides, fatty acid glutamates, α-olefinsulfonates, ether carboxylic acids, alkyl oligoglucosides, fatty acid glucamides, alkylamidobetaines, amphoacetals and/or protein fatty acid condensates, the latter specifically based on wheat proteins.

Oil bodies Bodycare compositions, such as creams, lotions and milks, usually comprise a series of further oil bodies and emollients which contribute to further optimizing the sensory properties. The oil bodies are usually present in a total amount of 1-50% by weight, specifically 5-25% by weight and in particular 5-15% by weight. As further oil bodies come, for example, Guerbet alcohols based on fatty alcohols having 6 to 18, specifically 8 to 10, carbon atoms, esters of linear C₆-C₂₂-fatty acids with linear or branched C₆-C₂₂-fatty alcohols or esters of branched C₆-C₁₃-carboxylic acids with linear or branched C₆-C₂₂-fatty alcohols, such as e. g. myristyl myristate, myristyl palmitate, myristyl stearate, myristyl isostearate, myristyl oleate, myristyl behenate, myristyl erucate, cetyl myristate, cetyl palmitate, cetyl stearate, cetyl isostearate, cetyl oleate, cetyl behenate, cetyl erucate, stearyl myristate, stearyl palmitate, stearyl stearate, stearyl isostearate, stearyl oleate, stearyl behenate, stearyl erucate, isostearyl myristate, isostearyl palmitate, isostearyl stearate, isostearyl isostearate, isostearyl oleate, isostearyl behenate, isostearyl oleate, oleyl myristate, oleyl palmitate, oleyl stearate, oleyl isostearate, oleyl oleate, oleyl behenate, oleyl erucate, behenyl myristate, behenyl palmitate, behenyl stearate, behenyl isostearate, behenyl oleate, behenyl behenate, behenyl erucate, erucyl myristate, erucyl palmitate, erucyl stearate, erucyl isostearate, erucyl oleate, erucyl behenate and erucyl erucate. Also of suitability are esters of linear C₆-C₂₂-fatty acids with branched alcohols, in particular 2-ethylhexanol, esters of C₁₈-C₃₈-alkylhydroxycarboxylic acids with linear or branched C₆-C₂₂-fatty alcohols, in particular dioctyl malate, esters of linear and/or branched fatty acids with polyhydric alcohols (such as e. g. propylene glycol, dimerdiol or trimertriol) and/or Guerbet alcohols, triglycerides based on C₆-C₁₀-fatty acids, liquid mono-/di-/triglyceride mixtures based on C₆-C₁₈-fatty acids, esters of C₆-C₂₂-fatty alcohols and/or Guerbet alcohols with aromatic carboxylic acids, in particular benzoic acid, esters of C₂-C₁₂-dicarboxylic acids with linear or branched alcohols having 1 to 22 carbon atoms or polyols having 2 to 10 carbon atoms and 2 to 6 hydroxyl groups, vegetable oils, branched primary alcohols, substituted cyclohexanes, linear and branched C₆-C₂₂-fatty alcohol carbonates, such as e. g. dicaprylyl carbonate (Cetiol® CC), Guerbet carbonates based on fatty alcohols having 6 to 18, specifically 8 to 10, carbon atoms, esters of benzoic acid with linear and/or branched C₆-C₂₂-alcohols (e. g. Finsolv® TN), linear or branched, symmetrical or asymmetrical dialkyl ethers having 6 to 22 carbon atoms per alkyl group, such as e. g. dicaprylyl ether (Cetiol® OE), ring-opening products of epoxidized fatty acid esters with polyols.

Fats and waxes Fats and waxes are added to the bodycare products as care substances and also in order to increase the consistency of the cosmetics. Typical examples of fats are glycerides, i. e. solid vegetable or animal products, which consist essentially of mixed glycerol esters of higher fatty acids. Fatty acid partial glycerides, i. e. technical-grade mono- and/or diesters of glycerol with fatty acids having 12 to 18 carbon atoms, such as, for example, glycerol mono/dilaurate, -palmitate or -stearate are also suitable for this purpose. Suitable waxes are, inter alia, natural waxes, such as e. g. candelilla wax, carnauba wax, japan wax, esparto grass wax, cork wax, guaruma wax, rice germ oil wax, sugar cane wax, ouricury wax, montan wax, beeswax, shellac wax, spermaceti, lanolin (wool wax), uropygial grease, ceresine, ozokerite (earth wax), petrolatum, paraffin waxes, microwaxes; chemically modified waxes (hard waxes), such as e. g. montan ester waxes, sasol waxes, hydrogenated jojoba waxes, and also synthetic waxes, such as e. g. polyalkylene waxes and polyethylene glycol waxes. Besides the fats, suitable additives are also fat-like substances such as lecithins and phospholipids. Examples of natural lecithins which may be mentioned are the cephalins, which are also referred to as phosphatidic acids and are derivatives of 1,2-diacyl-sn-glycerol-3-phosphoric acids. By contrast, phospholipids are usually understood to mean mono- and specifically diesters of phosphoric acid with glycerol (glycerol phosphates), which are generally classed as fats. In addition, sphingosines and sphingolipids are also suitable.

Suitable thickeners are, for example, Aerosil grades (hydrophilic silicas), polysaccharides, in particular xanthan gum, guar guar, agar agar, alginates and tyloses, carboxymethylcellulose and hydroxyethyl- and hydroxypropylcellulose, polyvinyl alcohol, polyvinylpyrrolidone and bentonites such as e. g. Bentone® Gel VS-5PC (Rheox).

UV light protection factors are to be understood as meaning, for example, organic substances (light protection filters) that are present in liquid or crystalline form at room temperature and which are able to absorb ultraviolet rays and release the absorbed energy again in the form of longer-wave radiation, e. g. heat. UV-B filters can be oil-soluble or water-soluble. Suitable typical UV-A filters are in particular derivatives of benzoylmethane. The UV-A and UV-B filters can of course also be used in mixtures, e. g. combinations of the derivatives of benzoylmethane, e. g. 4-tert-butyl-4′-methoxydibenzoylmethane (Parsol® 1789) and 2-ethylhexyl 2-cyano-3,3-phenylcinnamate (Octocrylene), and esters of cinnamic acid, specifically 2-ethylhexyl 4-methoxycinnamate and/or propyl 4-methoxycinnamate and/or isoamyl 4-methoxycinnamate. Combinations of this type are often combined with water-soluble filters such as e. g. 2-phenylbenzimidazole-5-sulfonic acid and the alkali metal, alkaline earth metal, ammonium, alkylammonium, alkanolammonium and glucammonium salts thereof.

As well as the specified soluble substances, insoluble light protection pigments, namely finely disperse metal oxides, are also suitable. Examples of suitable metal oxides are, in particular, zinc oxide and titanium dioxide. Besides the two aforementioned groups of primary light protection substances, it is also possible to use secondary light protection agents of the antioxidant type; these interrupt the photochemical reaction chain which is triggered when UV radiation penetrates into the skin.

Biogenic active ingredients are to be understood as meaning, for example, tocopherol, tocopherol acetate, tocopherol palmitate, ascorbic acid, (deoxy)ribonucleic acid and fragmentation products thereof, β-glucans, retinol, bisabolol, allantoin, phytantriol, panthenol, AHA acids, amino acids, ceramides, pseudoceramides, essential oils, plant extracts, such as e. g. prune extract, bambara nut extract and vitamin complexes.

Deodorizing active ingredients counteract, mask or eliminate body odors. Body odors arise as a result of the action of skin bacteria on apocrine perspiration, during which unpleasant smelling degradation products are formed. Accordingly, suitable deodorizing active ingredients are, inter alia, antimicrobial agents, enzyme inhibitors, odor absorbers or odor maskers.

Suitable insect repellents are, for example, N,N-diethyl-m-toluamide, 1,2-pentanediol or 3-(N-n-butyl-N-acetylamino)propionic acid ethyl ester), which is sold under the name Insect Repellent® 3535 by Merck KGaA, and also butylacetylaminopropionate.

A suitable self-tanning agent is dihydroxyacetone. Suitable tyrosine inhibitors, which prevent the formation of melanin and are used in depigmentation compositions, are, for example, arbutin, ferulic acid, kojic acid, coumaric acid and ascorbic acid (vitamin C).

Suitable preservatives are, for example, phenoxyethanol, formaldehyde solution, parabens, pentanediol or sorbic acid, and also the silver complexes known under the name Surfacine®, and the other substance classes listed in Annex 6, Parts A and B of the Cosmetics Ordinance.

Perfume oils which may be mentioned are mixtures of natural and synthetic fragrances.

Natural fragrances are extracts from flowers, stems and leaves, fruits, fruit peels, roots, woods, herbs and grasses, needles and branches, resins and balsams. Also of suitability are animal raw materials, such as, for example, civet and castoreum, and also synthetic fragrance compounds of the ester, ether, aldehyde, ketone, alcohol and hydrocarbon types.

Suitable pearlescent waxes, in particular for use in surface-active formulations, are for example: alkylene glycol esters, specifically ethylene glycol distearate; fatty acid alkanolamides, specifically coconut fatty acid diethanolamide; partial glycerides, specifically stearic acid monoglyceride; esters of polyhydric, optionally hydroxy-substituted carboxylic acids with fatty alcohols having 6 to 22 carbon atoms, specifically long-chain esters of tartaric acid; fatty substances, such as, for example, fatty alcohols, fatty ketones, fatty aldehydes, fatty ethers and fatty carbonates, which have in total at least 24 carbon atoms, specifically laurone and distearyl ether; fatty acids such as stearic acid, hydroxystearic acid or behenic acid, ring-opening products of olefin epoxides having 12 to 22 carbon atoms with fatty alcohols having 12 to 22 carbon atoms and/or polyols (without the sorbitan derivatives) having 2 to 15 carbon atoms and 2 to 10 hydroxyl groups, and mixtures thereof.

Superfatting agents which can be used are substances such as, for example, lanolin and lecithin, and also polyethoxylated or acylated lanolin and lecithin derivatives, polyol fatty acid esters, monoglycerides and fatty acid alkanolamides, the latter serving simultaneously as foam stabilizers.

Stabilizers which can be used are metal salts of fatty acids, such as e. g. stearates and ricinoleates of magnesium, aluminum and/or zinc.

To improve the flow behavior, hydrotropes, such as, for example, ethanol, isopropyl alcohol, or polyols, can also be used. Polyols which are suitable here specifically have 2 to 15 carbon atoms and at least two hydroxyl groups. The polyols can also contain further functional groups, in particular amino groups, and/or be modified with nitrogen.

In particular, preference is given to those cosmetic compositions which have an aqueous phase and an oil phase alongside one another and are present e. g. in the form of an emulsion (either water-in-oil, or oil-in-water) and which comprise, as one constituent, one or more isosorbide derivatives according to the above definition. Here, the isosorbide derivatives can be used as oil phase or emollient, or as a constituent of the oil phase. However, as explained in more detail below, they can also impart certain functional properties, depending on their structure.

Advantageously, e. g. esters of isosorbide are used within the context of the inventive teaching. Isosorbide esters can be synthesized by esterification processes known per se. By way of example, WO 01/83488 discloses a suitable method. Here, mono- or diesters of isosorbide, or mixtures of mono- and diesters, optionally in the presence of unmodified isosorbide, are possible and can be used within the context of the present teaching.

The monoesters of isosorbide can advantageously be, for example: isosorbide derivatives of the general formula (I) where: R═H and R′═COR″, where R″ is a linear or branched, saturated or unsaturated alkyl radical having 5 to 23 carbon atoms. Furthermore advantageous are also the diesters, e. g. those isosorbide derivatives of the general formula (I) for which: R and R′, independently of one another, are radicals COR″, where R″ is a linear or branched, saturated or unsaturated alkyl radical having 5 to 23 carbon atoms, where the linear, saturated alkyl radicals are particularly preferred.

Isosorbide derivatives of the general formula (I) which have been recognized as being particularly advantageous are those for which: R═H and R′ is a linear or branched alkyl radical having 6 to 22 carbon atoms. In the case of the diesters, particularly suitable derivatives are those isosorbide derivatives of the general formula (I) for which: R and R′ are, independently of one another, a linear or branched alkyl radical having 6 to 22 carbon atoms.

A further class of isosorbide derivatives is the hydroxyalkyl ethers. For these derivatives according to formula (I), R═H and R′ is a radical CH₂—CHOH—R″, where R′″ is a linear or branched alkyl radical having 6 to 22 carbon atoms. Here too, mono- and difunctional derivatives are possible: thus, it is also advantageously possible to use: isosorbide derivatives of the general formula (I) for which: R and R′, independently of one another, are a radical CH₂—CHOH—R″, where R′″ is a linear or branched alkyl radical having 6 to 22 carbon atoms.

A further class of isosorbide derivatives to be mentioned is the alkylene glycol derivatives, in particular the derivatives based on ethylene glycol: these are those isosorbide derivatives of the general formula (I) for which: R═H and R′ is a radical (CH₂—CHO)ₙ—, where n can mean an integer or fraction from 1 to 10. Continuing here too, again mono- or difunctional derivatives, or mixtures, optionally with underivatized isosorbide are possible. Thus, it is also advantageously possible to use those isosorbide derivatives of the general formula (I) for producing cosmetic compositions for which: R and R′, independently of one another, are a radical (CH₂—CHO)ₙ—, where n can be an integer or fraction from 1 to 10.

The next group of isosorbide derivatives is anionic derivatives. Here, mention is to be made primarily of the sulfates. Such isosorbide derivatives conform to the general formula (I) for which: R is a C6 to C22-alkyl radical and R′ is a radical SO₃X, where X represents a cation, specifically a monovalent cation and particularly a sodium or ammonium ion.

Mixtures of at least two structurally different isosorbide derivatives of the general formula (I) can of course be used alongside one another. It may be particularly advantageous to use those isosorbide derivatives of the general formula (I) in which either R or R′ is a hydrogen atom, thus the respective monofunctional derivatives of isosorbide, according to the above description, with particular emphasis being given to the monoesters.

Besides the differentiation as to whether mono- or difunctionalized derivatives are present, the structure and in particular the length of the alkyl chain of the isosorbide derivatives plays a particular importance for their suitability for producing cosmetic compositions: thus, in the case of the monoesters, advantageous derivatives are those in which the radical R″ is a linear, saturated alkyl radical having 10 to 18, in particular 11 to 17 and specifically 12 to 16, carbon atoms.

In the case of the diesters, the following derivatives are regarded as particularly advantageous: derivatives according to the general formula (I) where R and R′, independently of one another, are linear, saturated alkyl radicals having 10 to 18, in particular 11 to 17 and specifically 12 to 16, carbon atoms.

In the case of the monoethers, advantageous properties have been found if those derivatives according to the formula (I) are selected in which R′ is a linear or branched alkyl radical having 8 to 18, in particular 6 to 14, carbon atoms, specifically 8 to 14 carbon atoms and furthermore 10 to 14 carbon atoms.

In the case of the diethers: R and R′, independently of one another, are a linear or branched alkyl radical having 6 to 16 carbon atoms, specifically 8 to 14 carbon atoms and in particular 10 to 14 carbon atoms.

For the hydroxyalkyl ethers, two different structures have been recognized as being advantageous: firstly, derivatives according to the formula (I) are to be selected in which R′ is a radical CH₂—CHOH—R′″, where R′″ is a linear alkyl radical having 6 to 18, specifically 8 to 16, carbon atoms. Likewise suitable are the derivatives according to the general formula (I), in which R and R′, independently of one another, are a radical CH₂—CHOH—R′″, where R′″ is a linear alkyl radical having 6 to 18, specifically 8 to 16, carbon atoms.

In the case of the alkylene glycol derivatives, again the mono- and difunctionalized compounds according to the general formula (I) are suitable, where, as a consequence of production, predominantly to exclusively difunctionalized derivatives are present. Advantageously, R′ and R, independently of one another, are a radical (CH₂—CHO)ₙ—H, or (CH₂—CH(CH₃)O)ₘ—H, where n and m, independently of one another, can specifically be an integer or fraction from 1 to 4. As is customary for alkoxylates, pure ethoxylates, or pure propoxylates are possible, as are mixtures of ethoxylates and propoxylates with one another, it being possible for the distribution to be either randomized or blockwise.

As regards the amounts in which the isosorbide derivatives according to the above description are used in cosmetic compositions, this depends on the specific formulation and can vary over a wide range. Typical amounts, however, are 0.5 to 30% by weight, specifically in amounts of from 1 to 15% by weight and in particular in amounts of from 1.5 to 5% by weight, in each case based on the total weight of the cosmetic composition.

In one particular embodiment, the present invention relates to the use of the isosorbide derivatives for producing cosmetic compositions which are free from silicone oils.

The isosorbide derivatives are exceptionally suitable for producing aqueous cosmetic compositions and furthermore to those aqueous compositions which also comprise nonionic emulsifiers (without the isosorbide derivatives), hydrocarbons, astringents, dyes, fragrances, propellants, thickeners, and/or pearlizing agents.

Depending on their structure, the derivatives exhibit particularly positive properties which render them suitable for certain formulation tasks: isosorbide derivatives according to the general formula (I) can generally be used as thickeners, emulsifiers, as oil phase/emollient and/or as foam improver for aqueous cosmetic compositions.

The present invention further provides specific, selected derivatives of isosorbide: these are isosorbide derivatives according to the general formula (I)

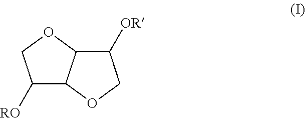

where R and R′, independently of one another, are a radical COR″, in which R″ is a linear, saturated alkyl radical having 5 to 11 and specifically 5 to 9, and in particular 5 to 7, carbon atoms. These isosorbide derivatives are especially suitable as emollient in cosmetic compositions.

A further selected isosorbide derivative conforms to the general formula (I)

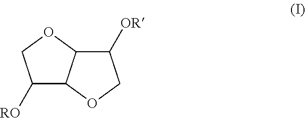

where one radical R or R′ is a hydrogen atom and the other radical R or R′ is a group COR″, in which R″ is a linear, saturated alkyl radical having 15 to 19 and specifically 15 to 17 carbon atoms. These derivatives are used advantageously in pearlescent compositions.

A further isosorbide derivative conforms to the general formula (I)

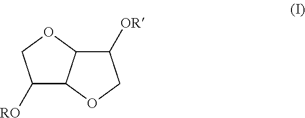

where one radical R or R′ is a hydrogen atom and the other radical R or R′ is a group COR″, in which R″ is a linear, saturated alkyl radical having 9 to 13 and specifically 10 to 11 carbon atoms. These derivatives are used advantageously as thickeners in aqueous cosmetic compositions.

Finally, preference is likewise given to the isosorbide derivative according to the general formula (I)

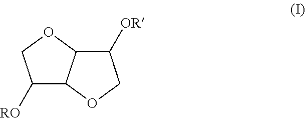

where R or R′ is a hydrogen atom and the other radical R or R′ is a group CH₂—CHOH—R″, in which R″ is a linear alkyl radical having 8 to 18, specifically 8 to 14, carbon atoms and furthermore also 12 to 18 or 12 to 14 carbon atoms. These derivatives can advantageously be used as emulsifier in cosmetic compositions, in which case particularly the derivatives in which R″ is a linear alkyl radical having 8 to 18, specifically 8 to 14 carbon atoms are to be selected.

Further subjects relate to the use of isosorbide derivative according to the general formula (I)

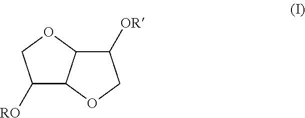

where R or R′ is a hydrogen atom and the other radical R or R′ is an alkyl group having 8 to 12 carbon atoms, as foam booster for aqueous compositions which comprise foaming substances.

Moreover, the use of isosorbide derivative according to the general formula (I)

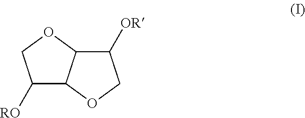

where R and R′ are a group (CH₂—CH₂—O)ₙ—H and/or (CH₂—CH(CH₃)—O)ₘ—H, where n and m, independently of one another, are an integer or fraction from 1 to 10, as foam booster for aqueous compositions which comprise foaming substances.

And also the use of isosorbide derivative according to the general formula (I)

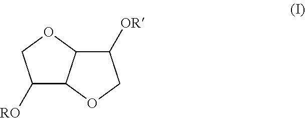

where one group R or R′ is an alkyl radical having 8 to 18 carbon atoms and the other group R or R′ is a radical SO₃X, where X represents a sodium or ammonium ion, as thickener for aqueous compositions, with the proviso that R≠R′.

The present teaching also provides cosmetic compositions which comprise at least one water phase and one oil phase, where they comprise at least one isosorbide derivative according to the general formula (I).

### Examples

The investigations, described below, relating to the properties of the isosorbide derivatives were carried out. Wherever ingredients are specified, the INCI nomenclature was used.

To determine the viscosity and assess the pearlescence, isosorbide esters with a high mono fraction were incorporated with 1% by weight into the formulation described below shown in Table 1 which comprised a dye for the purposes of better assessing the pearlescence. In this connection, the influence of the chain length was investigated.

TABLE 1
Formulation:

| Ingredient | Fraction in % by weight |
|---|---|
| Sodium Laureth Sulfate | 8.6 |
| NaCl | 2.5 |
| Coco-Glucoside | 1.6 |
| Cocamidopropyl Betaine | 1.4 |
| Test substance | 1.0 |
| Sicomet amaranth (1% strength aq.) | 0.1 |
| Euxyl K 400 | 0.1 |
| Water | ad 100 |

The viscosity was determined at 20° C. using the Brookfield RVT viscometer (spindle: RV spindle No. 5).

The pearlescence was assessed visually by comparison with a standard pearlizing agent (EGDS=Cutina AGS) and evaluated on a scale from 0 (no pearlescence) to 3 (pearlescence comparable with standard).

The mono content given in Table 2 below was determined by means of GC analysis. Missing fractions up to 100% are composed essentially of the diester and, in small amounts, of isosorbide and/or free fatty acid.

TABLE 2

| C chain length | Mono content/SI % | Viscosity/mPas | Pearlescence |
|---|---|---|---|
| 14 | 87 | 29 500 | 0 |
| 16 | 85 | 17 000 | 2 |
| 18 | 77 | 10 300 | 3 |

SI = surface integral

The measurements reveal a relationship between the viscosity and the chain length. The shorter-chain C14-ester again builds up a significantly higher viscosity than the longer-chain C16- and C18-esters. Pearlescence was observed only in the case of the longer-chain C16- and C16-esters. The C18-ester has a comparable pearlescent effect to the standard.

Saturated and unsaturated, linear and branched isosorbide derivatives with different chain lengths and functional groups (esters, ethers) were evaluated regarding their foam properties using the following formulation.

TABLE 3
Formulation Foam Test

| ingredient | % |
|---|---|
| Sodium Laureth Sulfate | 10.8 |
| Cocoamidopropyl Betaine | 1.5 |
| Coco-Glucoside | 1.5 |
| Sodium chloride (viscosity adjustment) | 0.9-2.5 |
| Isosorbide derivative | 1 |
| Sodium benzoate | 0.5 |
| Cationic polymer | 0.2 |
| Citric acid | 0.2 |
| water | Up to 100 |

All derivatives have a mono content higher than 75%. Typical by-products are the corresponding difunctionalized products, remaining starting material (isosorbide) or fatty alcohols (only for ethers).

### Performance Test: Foam Behavior

The foam behavior of four esters and two ethers of isosorbide was measured. All formulations were diluted with hard water (ratio 1:4, 30° C.) and stirred in a beaker for 10 s at 2000 rpm before the foam high was determined. All results were compared, also to a placebo formulation. All measurements were repeated triply and the results were averaged.

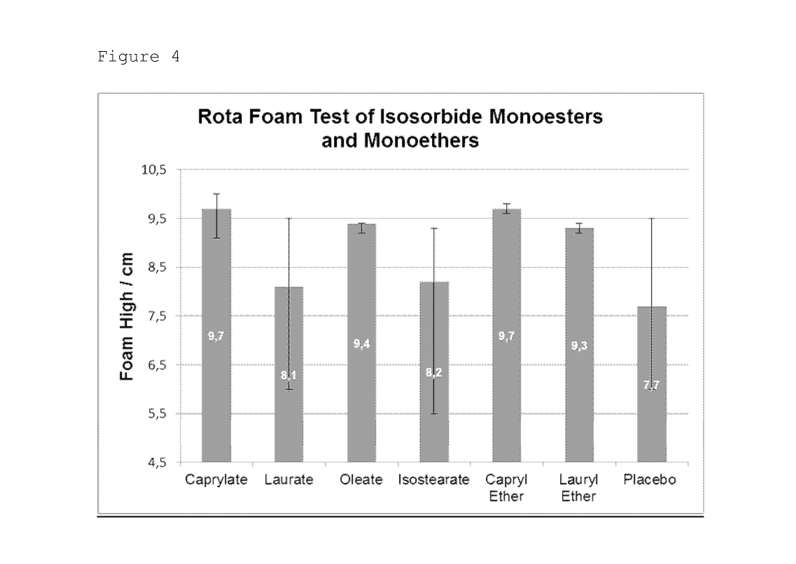

The results have been obtained in a Rota Foam Test and visualized in the diagram of FIG. 4. As can be seen the average amount of foam generated of a formulation containing isosorbide esters or ethers is higher compared to a placebo formulation without additive. The best performance could be observed for shorter chain (capryl ester and ether, lauryl ether) and linear unsaturated derivatives (oleate).

Furthermore, the properties of isosorbide ether sulfates were investigated: to determine the foam height, aqueous solutions with an active content of the substance to be tested of 2.5% by weight were prepared. Sodium isosorbide lauryl ether sulfate was compared with the standard anionic surfactant sodium laureth sulfate and nonionic lauryl glucoside.

### Carrying Out the Foam Measurement:

The formulations were prepared with 400 g; in each case, 100 g of the formulation were foamed after heating to 30° C. Foaming was effected in an 800 ml beaker for 10 sec at 2000 revolutions using a Meiser disk. The foam height was determined in the beaker. A four-fold determinations were carried out and then averaged.

| Surfactant | Foam height/cm |
|---|---|
| Sodium Laureth Sulfate | 9.1 |
| Isosorbide based EO-free Sulfate (89% AS): | 7.3 |
| R = C₁₂H₂₅, R′ = SO₃Na | |
| Lauryl Glucoside | 6.0 |

AS = active substance

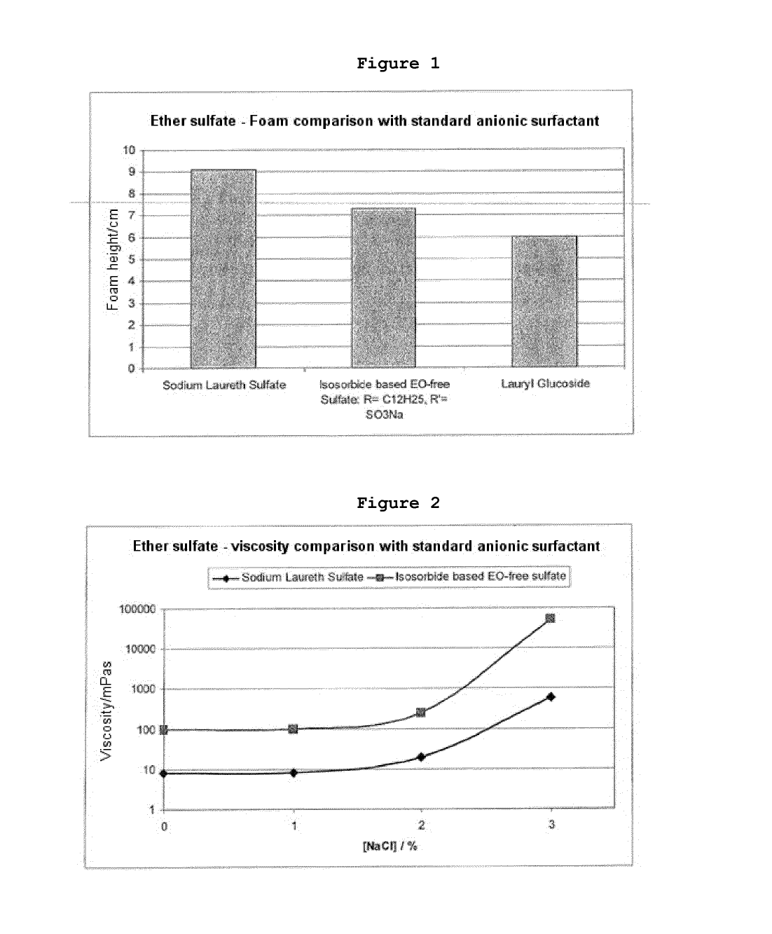

The results can be found in FIG. 1: the foaming ability of the EO-free isosorbide ether sulfate is at a high level, even though a lower foam height is reached than with sodium laureth sulfate.

Determination of the Thickening with NaCl:

To determine the thickenability with sodium chloride, 12% strength aqueous solutions of the test substances with different sodium chloride concentrations were prepared and measured at 22° C. using a Brookfield, DV-II+Pro viscometer (spindle LV62 and 60 revolutions for low viscosities, spindle LV64 and 6 revolutions for high viscosities).

| | Viscosity/mPas at [NaCl] | | | |
|---|---|---|---|---|
| Anionic surfactant | 0% | 1% | 2% | 3% |
| Sodium Laureth Sulfate | 8 | 8 | 19 | 590 |
| Isosorbide based EO-free Sulfate (89% AS): | 100 | 100 | 250 | 52 589 |
| R = C₁₂H₂₅, R′ = SO₃Na | | | | |

The results are shown in FIG. 2. Aqueous solutions which comprise isosorbide ether sulfate can be thickened much more easily with sodium chloride and have a higher starting viscosity. The built-up viscosity is up to two orders of magnitude above that which is achieved with sodium laureth sulfate under identical conditions.

In another experiment, saturated and unsaturated, linear and branched isosorbide derivatives with different chain lengths and functional group (esters, ethers) were evaluated regarding their thickening ability in the presence of sodium chloride using the following formulation.

TABLE 4
Formulation Thickening Test

| ingredient | % |
|---|---|
| Sodium Laureth Sulfate | 10.8 |
| Cocoamidopropyl Betaine | 1.5 |
| Coco-Glucoside | 1.5 |
| Sodium chloride | 0.5, |
| | 1, |
| | 1.5, |
| | 2 |
| Isosorbide derivative | 1 |
| Sodium benzoate | 0.5 |
| Cationic polymer | 0.2 |
| Citric acid | 0.2 |
| water | Up to 100 |

For each test substance four formulations with different sodium chloride concentrations were prepared. All derivatives have a mono content higher than 75%. Typical by-products are the corresponding difunctionalized products, remaining starting material (isosorbide) or fatty alcohols (only for ethers).

The viscosity of all formulations was measured at 20° C. using a Brookfield RVF viscometer (spindle number 3-5, 20 rpm).

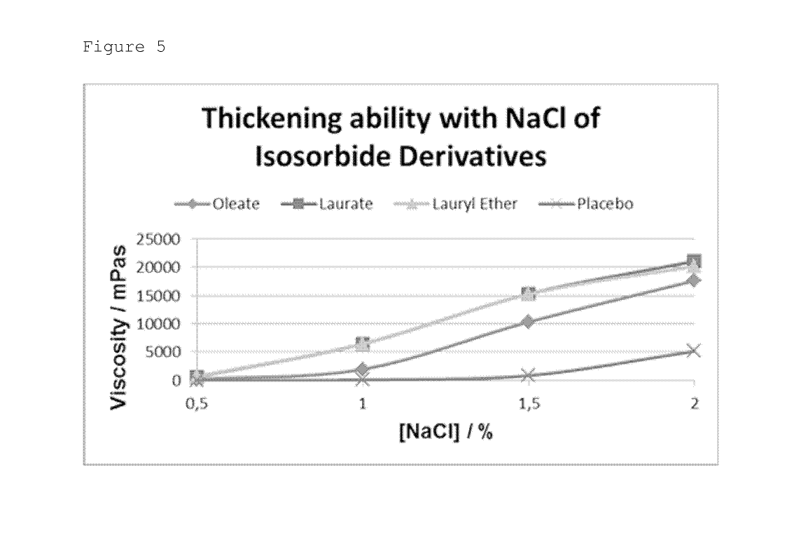

The thickening ability of these isosorbide monoesters and monoethers has been tested. The results are shown in FIG. 5. As can be seen all tested derivatives show a very good thickening performance. Especially the lauryl ester and ether are able to thicken the test formulation already at low sodium chloride concentration.

Various isosorbide samples with a different degree of alkylation (ethoxylated or propoxylated) were assessed in the following formulation according to Table 5 with regard to their foaming ability compared to a placebo formulation (without additive). The results are given in Table 6.

TABLE 5
Formulations:

| Ingredient | Fraction in % by wt. |
|---|---|
| Sodium Laureth Sulfate | 10.8 |
| NaCl | 2.4 |
| Test substance | 2 |
| Coco-Glucoside | 1.5 |
| Dist. water | ad 100 |

TABLE 6

| Test substance | Foam height/cm |
|---|---|
| Isosorbide + 4 EO | 8.2 |
| Isosorbide + 8 EO | 8.4 |
| Isosorbide + 12 EO | 7.7 |
| Isosorbide + 4 EO + 2 PO | 7.8 |
| Isosorbide + 8 EO + 2 PO | 8.6 |
| Isosorbide + 12 EO + 2 PO | 8.6 |
| Placebo | 6.0 |

Carrying Out the Foam Measurement:

For the formulations, the individual components were weighed in successively and mixed together. They were then diluted 1:4 with hard water and foamed at 30° C. using a Meiser disk in an 800 ml beaker at 2000 revolutions for 10 s. The foam height was measured in the beaker. All of the products exhibited a significant improvement in foam height compared with the formulation without additive.

Improvement in Foam Quality:

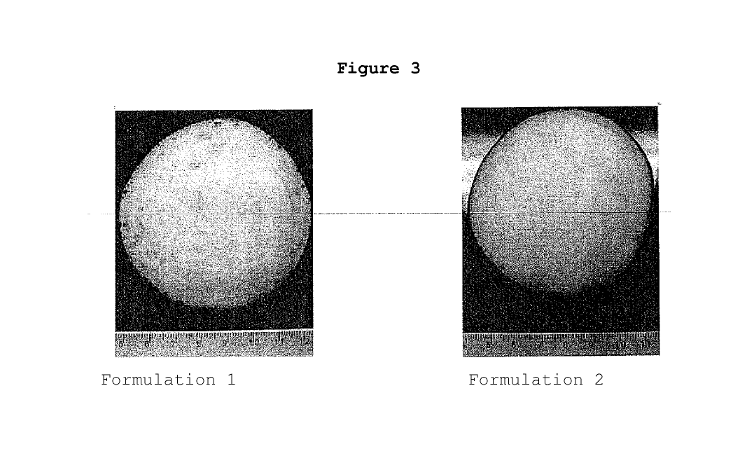

To assess the foam quality, the following two formulations were prepared, which were then assessed as regards their foam quality in a foam test visually (see photos in FIG. 3) by comparison with internal standards:

Formulation 1 (placebo):

| Ingredient | Fraction in % by wt. |
|---|---|
| Sodium Laureth Sulfate | 9 |
| Coco-Glucoside | 3 |
| Dist. water | ad 100 |

Formulation 2 (placebo):

| Ingredient | Fraction in % by wt. |
|---|---|
| Sodium Laureth Sulfate | 9 |
| Coco-Glucoside | 3 |
| ISB + 4 EO | 2 |
| Dist. water | ad 100% |

Carrying Out the Foam Measurement:

For the formulations, the individual components were weighed in successively and mixed together. Then, 17.9 g of the formulation are made up to 100 g with hard water, to give a 2.5% strength solution. The 2.5% strength solution was impacted using a Meiser disk in an 800 ml beaker at 2000 revolutions for 10 s. The foam height was measured in the beaker. The foam was spooned onto a Ceran plate and photographed again. With formulation 1, which comprises no isosorbide ethoxylate, only a qualitatively low quality foam can be produced compared to formulation 2. Accordingly, additives of isosorbide ethoxylate increase the foam quality to a considerable extent.

1. A method of producing a cosmetic composition, the method comprising mixing a cosmetic ingredient with an isosorbide derivative according to the general formula (I) 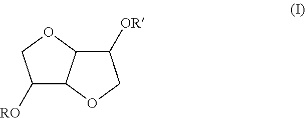
wherein R and R′, independently of one another, are:
(i) a hydrogen atom, or
(ii) a radical COR″, where R″ is a linear or branched, saturated or unsaturated alkyl radical having 5 to 23 carbon atoms, or
(iii) a linear or branched, saturated or unsaturated alkyl radical having 6 to 22 carbon atoms, or
(iv) a radical CH₂—CHOH—R′″, where R′″ is a linear or branched alkyl radical having 6 to 22 carbon atoms, or
(v) a radical (CH₂—CH₂O)ₙ—H and/or (CH₂—CH(CH₃)—O)ₘ—H and/or CH₂—CHOH—R′″, where n and m, independently of one another, can be an integer or fraction from 1 to 10, or
(vi) for a radical SO₃X, where X represents a sodium or ammonium ion,
with the proviso that at most one of the radicals R and R′ is a hydrogen atom.

2. The method of claim 1, wherein
R═H and R′═COR″, wherein R″ is a linear or branched alkyl radical having 5 to 23 carbon atoms.

3. The method of claim 1, wherein
R and R′, independently of one another, are radicals COR″, wherein R″ is a linear or branched, saturated or unsaturated alkyl radical having 5 to 23 carbon atoms.

4. The method of claim 1, wherein
R═H and R′ is a linear or branched alkyl radical having 6 to 22 carbon atoms.

5. The method of claim 1, wherein
R and R′, independently of one another, are a linear or branched alkyl radical having 6 to 22 carbon atoms.

6. The method of claim 1, wherein
R═H and R′ is a radical CH₂—CHOH—R′″, wherein R′″ is a linear or branched alkyl radical having 6 to 22 carbon atoms.

7. The method of claim 1, wherein
R and R′, independently of one another, are a radical CH₂—CHOH—R′″, wherein R′″ is a linear or branched alkyl radical having 6 to 22 carbon atoms.

8. The method of claim 1, wherein
R═H and R′ is a radical (CH₂—CHO))ₙ— and/or (CH₂—CH(CH₃)—O)ₘ—, wherein n and m, independently of one another, are an integer or fraction from 1 to 10.

9. The method of claim 1, wherein R and R′, independently of one another, are a radical (CH₂—CHO)ₙ—, where n is an integer or fraction from 1 to 10, or are a radical (CH₂—CH(CH₃)—O)ₘ—, where m is an integer or fraction from 1 to 10, or R and R′ contain both radicals (CH₂—CHO)ₙ and radicals (CH₂—CH(CH₃)—O)ₘ alongside one another.

10. The method of claim 1, wherein
R is a C6 to C22-alkyl radical and R′ is a radical SO₃X, wherein X represents a sodium or ammonium ion.

11. The method of claim 1, wherein at least two structurally different isosorbide derivatives of the general formula (I) are mixed alongside one another with the cosmetic ingredient.

12. The method of claim 1, wherein either R or R′ is a hydrogen atom.

13. The method of claim 2, wherein R″ is a linear, saturated alkyl radical having 10 to 18 carbon atoms.

14. The method of claim 3, wherein R and R′, independently of one another, are linear, saturated alkyl radicals having 10 to 18 carbon atoms.

15. The method of claim 4, wherein R′ is a linear or branched alkyl radical having 6 to 12 carbon atoms.

16. The method of claim 5, wherein R and R′, independently of one another, are a linear or branched alkyl radical having 6 to 12 carbon atoms.

17. The method of claim 6, wherein R′ is a radical CH₂—CHOH—R′″, wherein R′″ is a linear alkyl radical having 6 to 12.

18. The method of claim 7, wherein R and R′, independently of one another, are a radical CH₂—CHOH—R′″, wherein R′″ is a linear alkyl radical having 6 to 12.

19. The method of claim 8, wherein R′ is a radical (CH₂—CHO)ₙ—, wherein n is an integer or fraction from 1 to 4.

20. The method of claim 9, wherein R and R′, independently of one another, are a radical (CH₂—CHO)ₙ—, wherein n is an integer or fraction from 1 to 4.

21. The method of claim 1, wherein the isosorbide derivative comprises from 0.5 to 30% by weight of the cosmetic composition.

22. The method of claim 1, wherein the cosmetic composition is free from silicone oils.

23. The method of claim 1, wherein the cosmetic composition is an aqueous cosmetic composition and wherein the cosmetic ingredient is selected from nonionic emulsifiers, hydrocarbons, astringents, dyes, fragrances, propellants, thickeners, and/or pearlizing agents.

24. The method of claim 1, wherein the cosmetic composition is aqueous, and wherein the isosorbide derivative is effective to thicken the aqueous cosmetic composition.

25. The method of claim 1, wherein the cosmetic composition is aqueous and wherein the isosorbide derivative is effective to emulsify the aqueous cosmetic composition.

26. The method of claim 1, wherein the cosmetic composition is aqueous, and wherein the isosorbide derivative is effective to form an oil phase in the aqueous cosmetic composition.

27. The method of claim 1, wherein the cosmetic composition is aqueous, and wherein the isosorbide derivative is effective to improve foam in the aqueous cosmetic composition.

28. An isosorbide derivative according to the general formula (I) 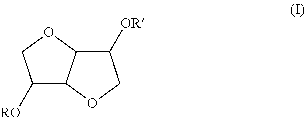
wherein R and R′, independently of one another, are a radical COR″, wherein R″ is a linear, saturated alkyl radical having 5 to 11 carbon atoms.

29. A method of preparing a cosmetic composition, the method comprising mixing a cosmetic ingredient and an emollient comprising the isosorbide derivative of claim 28.

30. An isosorbide derivative according to the general formula (I) 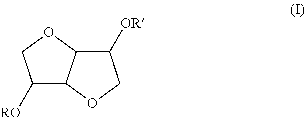
wherein one radical R or R′ is a hydrogen atom and the other radical R or R′ is a group COR″, wherein R″ is a linear, saturated alkyl radical having 15 to 19 carbon atoms.

31. A method of preparing pearlescent compositions, the method comprising mixing a cosmetic ingredient and the isosorbide derivative of claim 30.

32. An isosorbide derivative according to the general formula (I) 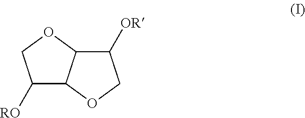
wherein one radical R or R′ is a hydrogen atom and the other radical R or R′ is a group COR″, wherein R″ is a linear, saturated or unsaturated alkyl radical having 9 to 15.

33. A method of preparing aqueous cosmetic compositions, the method comprising mixing a cosmetic ingredient and a thickener comprising the isosorbide derivative of claim 32.

34. An isosorbide derivative according to the general formula (I) 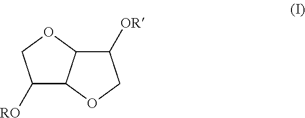
wherein R or R′ is a hydrogen atom and the other radical R or R′ is a group CH₂—CHOH—R″, wherein R″ is a linear alkyl radical having 12 to 18 carbon atoms.

35. A method of preparing cosmetic compositions, the method comprising mixing a cosmetic ingredient and an emulsifier comprising the isosorbide derivative of claim 34.

36. A method of boosting the foam of an aqueous composition, the method comprising mixing a foaming substance with a foam booster comprising an isosorbide derivative according to the general formula (I) 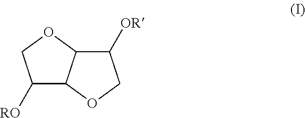
wherein R or R′ is a hydrogen atom and the other radical R or R′ is an alkyl group having 8 to 12 carbon atoms.

37. A method of boosting the foam of an aqueous composition, the method comprising mixing a foaming substance with a foam booster comprising an isosorbide derivative according to the general formula (I) 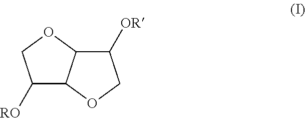
wherein R and R′ are a group ((CH₂—CH₂—O)ₙ—H and/or (CH₂—CH(CH₃)—O)ₘ—H, wherein n and m, independently of one another, are an integer or fraction from 1 to 10.

38. A method of thickening an aqueous composition, the method comprising, mixing an aqueous composition and a thickener comprising an isosorbide derivative according to the general formula (I) 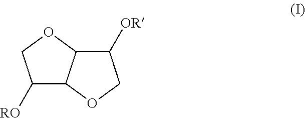
wherein one group R or R′ is an alkyl radical having 8 to 18 carbon atoms and the other group R or R′ is a radical SO₃X, with the proviso that R≠R′.

39. A cosmetic composition comprising at least one water phase and one oil phase, and the isosorbide derivative of claim 28.
